(19) United States
(12) Patent Application Publication
(10) Pub. No.: US 2005/0256524 A1
(43) Pub. Date: Nov. 17, 2005

(54) RF ablation device and method of use

(76) Inventors: Gary L. Long, Cincinnati, OH (US); Omar J. Vakharia, Mason, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/127,512

(22) Filed: May 12, 2005

Related U.S. Application Data
(60) Provisional application No. 60/571,225, filed on May 14, 2004.

(51) Int. Cl. A61B 18/14

(52) U.S. Cl. 606/41

(57) ABSTRACT

An ablation device is provided. Electrodes are supported on counter rotating rollers. The rollers can be rotated to provide a desired spacing between the two electrodes. The rollers and electrodes can be associated with a side opening in a housing supported at a distal end of a flexible, elongated overtube sized to receive an endoscope.

### CROSS-REFERENCES TO RELATED APPLICATIONS

This application incorporates by reference and claims priority to U.S. Provisional Patent Application Ser. No. 60/571,225 filed May 14, 2004, “Improvement to RF Ablation Device and Method of Use.

This application cross references and incorporates by reference U.S. patent application Ser. No. 10/394,285 filed Mar. 21, 2003; U.S. patent application Ser. No. 10/105,609 filed Mar. 25, 2002; U.S. patent application Ser. No. 10/105, 610 filed Mar. 25, 2002; and U.S. patent application Ser. No. 10/105,722 filed Mar. 25, 2002.

### FIELD OF THE INVENTION

The present invention is directed to medical devices in general, and more particularly to medical devices for ablation.

### BACKGROUND

US Patent Application Publication no. U.S. 2003/0216727 A1 dated Nov. 20, 2003 describes a medical device for ablation or removal of tissue by surgical means in the esophagus and GI tract of a patient, particularly for patients having chronic gastro-esophageal reflux disease (GERD). GERD can cause the the inner lining of the esophagus to change from squamous mucosa to columnar mucosa in a condition commonly known as Barrett's esophagus. Barrett's tissue can be ablated using radio frequency (RF) energy. Varices, which are dilated blood vessels, similar in appearance to varicose veins, may be caused by liver cirrhosis or portal hypertension. Varices, which tend to be mostly axial in orientation along the esophageal wall, may also be treated with RF energy. RF energy ablation for varices is the primary interest of the present patent application.

Published Application U.S. 2003/0216727 A1 describes an ablation device having a window and electrodes connected to an RF energy source. The device can be used to ablate the tissue. The ablated tissue then sloughs off over time from the body because the cauterized tissue lacks a blood supply. A vacuum can be applied (e.g. such as through an endoscope disposed within the ablation device) to help ensure proper tissue contact with respect to the window and the electrodes.

Still, scientists and engineers in the medical arts continue to seek new and improved methods for treatment of tissue.

### SUMMARY OF THE INVENTION

The present invention provides a device and method for treating tissue. In one embodiment, the medical device includes a first electrode and a second electrode. The first electrode can be movable, such as by rotation, relative to the second electrode to provide a predetermined desired spacing between an edge of the first electrode and an edge of the second electrode. Each of the first and second electrodes can be rotatable, such as by being supported on rotating members which engage tissue and which provide a desired predetermined orientation and spacing of the first and second electrodes.

In one embodiment, a first electrode is supported on a first tissue engaging roller, and a second electrode is supported on a second tissue engaging roller. The first and second rollers can be supported and driven to be counter rotating with respect to each other. Each of the rollers can include a feature, such as a plurality of protrusions, for engaging tissue between the rollers as the rollers are rotated. The first and second rollers can be supported in a housing disposed at the distal end of an elongate, flexible overtube. The housing can include a side opening through which tissue can be engaged by the counter rotating rollers prior to energizing the electrodes. After the tissue is treated, the directions of rotation of the rollers can be reversed to release the tissue. An endoscope can be positioned in one or both of the overtube and the housing.

The present invention also provides a method for treating tissue. In one embodiment, the method comprises the steps of providing a first tissue engaging member having a first electrode; providing a second tissue engaging member having a second electrode; moving at least one of the tissue engaging members to provide a desired spacing between the first and second electrodes; and energizing the electrodes to treat the tissue.

In one embodiment of the present invention, tissue to be treated is mechanically gathered and lifted away from the tissue site (such as the esophageal wall) and ablated with electrodes associated with the apparatus for mechanically gathering the tissue.

In one embodiment, the present invention provides an opening in an ablation cap or overtube, so that varix tissue may be mechanically drawn into the cap or overtube prior to applying RF energy with electrodes.

In one embodiment, the present invention provides a mechanical device for mechanically gathering the varix and lifting it away from the esophageal wall into the aperture, so that the varix tissue may be ablated at the submucosal level without having to burn through the entire varix, thereby providing more efficient ablation and requiring a relatively small amount/low level of energy, to thereby minimize unintentional ablation of surrounding tissue

According to one embodiment, the mechanical gathering of tissue can be provided by a mechanical device comprising a pair of parallel “D-shaped” rollers pivoted from just inside an opening in an overtube or endcap. The rollers can be disposed for rotation about generally parallel axes, and the rollers can be disposed at either side of the opening. Upon contact with a portion of varix at the opening, the rollers can each be rotated between about 90° to about 180° in opposing directions such that tissue is frictionally gripped and pulled into the aperture. The rollers can include bumps, ridges, or other surface features to better grip tissue. A generally rectangular plate electrode can be disposed on the “flat” portion of each D-shaped roller. The roller can provide planar alignment of the two electrodes at the end of the roller rotation step, such that the electrodes are substantially parallel, and such that the electrode plates are positioned tangent to the outer surface of the cap or overtube at the sides of the tissue receiving aperture. RF electrical energy is then provided (such as by pulsing) through the bipolar electrodes to ablate the varix tissue between the electrodes.

### BRIEF DESCRIPTION OF THE DRAWINGS

FIG. 1 is a top plan view of an ablation device and showing a housing attachable to a distal end of an endoscope, the housing having a pair of rollers partially extending through an aperture therein.

FIG. 2 is a front elevation view of a varix ablation device of FIG. 1.

FIG. 3 is a cross-sectioned top plan view of the housing of FIG. 1, taken along section line 3-3 of FIG. 2, showing a small servomotor coupled to a gear shaft to drive the pair of rollers.

FIG. 4 is a partial top view similar to FIG. 3, showing a flexible cable instead of a motor coupled to the gear shaft to drive the pair of rollers.

FIG. 5 is a cross-sectioned elevation view, taken along section line 5-5 of FIG. 1, showing drive gears for the pair of rollers, and the rollers in a position for device insertion into a patient.

FIG. 6 is a cross-sectioned view similar to FIG. 5, showing the rollers rotated 180 degrees to a position where flat electrodes on the rollers are positioned for tissue ablation.

FIG. 7 is a cross-sectioned elevation view, taken along section line 7-7 of FIG. 1, showing the housing pressed against a protruding varix in an esophagus of a patient.

FIG. 8 is a cross-sectioned elevation view similar to FIG. 7, showing the rollers rotated 180 degrees to gather tissue between them and to align electrodes for RF energy actuation.

FIG. 9 is a cross-sectioned elevation view similar to FIG. 8, showing ablated tissue after RF energy actuation.

FIG. 10 is a cross-sectioned elevation view similar to FIG. 9, showing the rollers retracted 180 degrees as the device is moved away from the esophageal wall to leave the ablated varix intact, where it will eventually slough off.

FIG. 11 is a perspective view of an alternative means for driving roller gears 180 degrees, showing a single helix worm inserted into a spur gear and linearly actuated back and forth by square splines to cause the gear to rotate.

FIG. 12 is a perspective view of a varix ablation device positioned within a body cavity, showing a user's hands holding an endoscope and said varix ablation device.

### DETAILED DESCRIPTION OF THE INVENTION

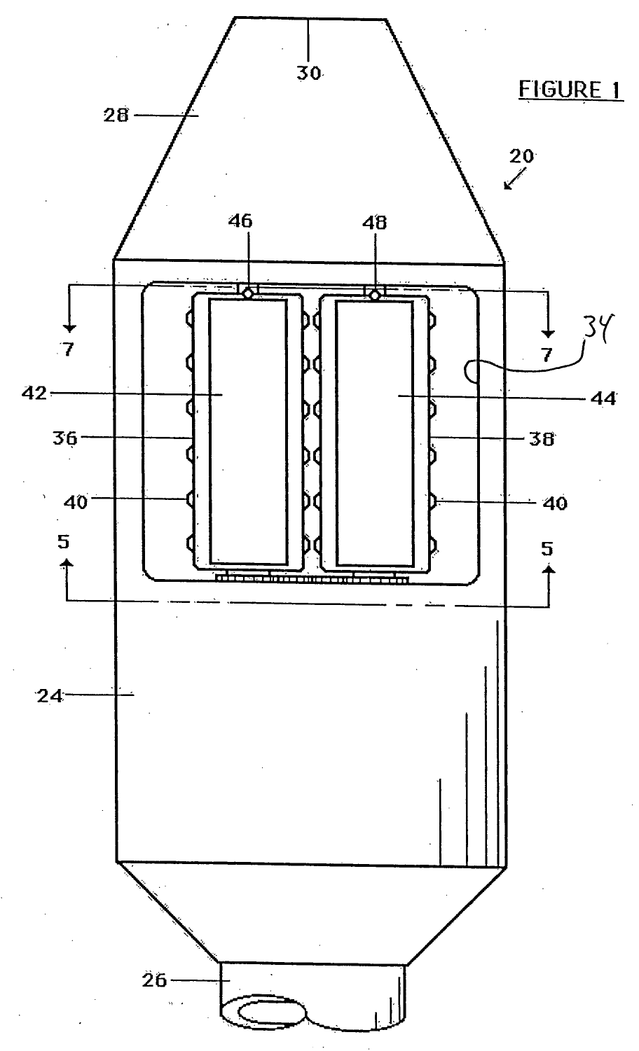

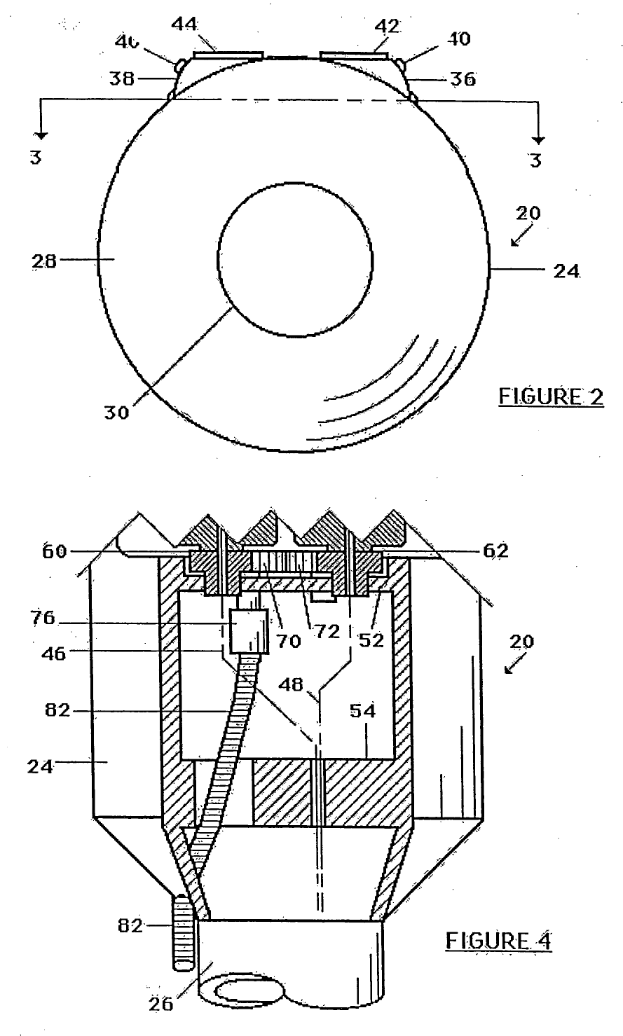

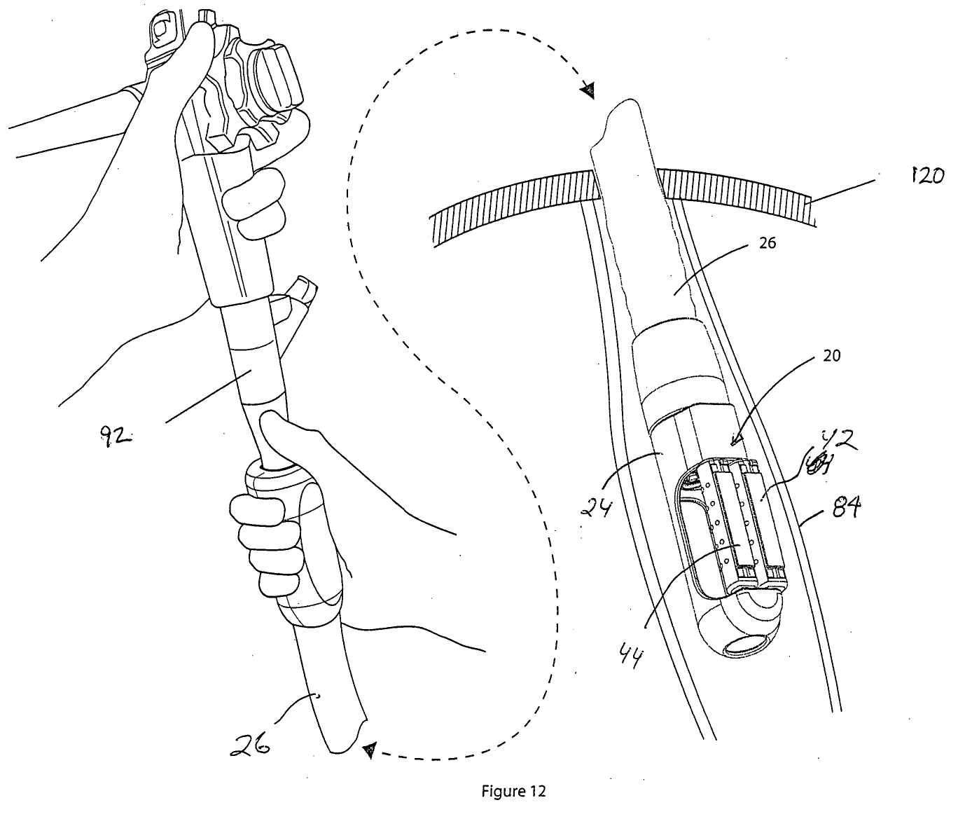

With reference to FIGS. 1 and 2, there is shown a tissue ablation device 20 according to one embodiment of the present invention. Device 20 can be releasably or permanently attached to the distal end of a commercially available endoscope, such as a model GIF-P140 made by Olympus America Inc. of Melville, N.Y. having an outside diameter of about 8 mm. For instance, device 20 can include a semi-rigid cylindrical housing 24 which is attached to the endoscope perimeter by any suitable means for connecting one cylinder to another along colinear longitudinal axes. Shrink wrap polymer may be added to make such connection leak-tight, as is also commonly known in the art. Alternatively, housing 24 may be disposed at a distal end of a relatively long, flexible elongated overtube 26 (e.g. see FIG. 12). Overtube 26 may be formed of corrugated plastic tubing, which passes along the outer surface of the endoscope and out of the patient's body so that housing 24 may be rotated about the endoscope and/or slid longitudinally over the endoscope.

Housing 24 can have a conical distal member 28 which provides for a smooth entry of ablation device 20 into the alimentary canal of a patient. Distal member 28 can have an opening 30 sized about 8.5 mm in diameter to allow an endoscope to pass through, and through which unobstructed camera vision of the inside of the patient's alimentary canal is obtained from a light and camera within the endoscope. In one embodiment, conical member 28 is made of flexible polymer, such as polyvinylchloride (PVC), polyethylene terephthalate (PET), etc., and it is attached to housing 24 by any suitable means, including without limitation by threading, polymer welding, snap-fit, or other suitable means known in the art.

Housing 24 can have an outer diameter of about 20 mm and a length of about 40 mm. Housing 24 can be made of a transparent polymer, such as polycarbonate. Housing 24 can also include a side opening which can be a generally rectangular tissue receiving aperture 34 along one side, which aperture can be about about 24 mm long and about 18 mm wide as measured along the circumferential direction around housing 24.

Two generally parallel rollers 36 and 38 can be disposed within housing 24, such as by being positioned just inside aperture 34. Each of the generally parallel rollers 36 and 38 can have a “D-shape” cross section. Rollers 36 and 38 can be formed of an electrically and thermally insulating material, such as nylon. The rollers 36 and 38 can have substantially the same size and shape. Each roller can have a length of about 21 mm and a root diameter of about 6 mm. Rollers 36 and 38 can each have hemispherical protrusions 40 which extend about 0.75 mm from roller surfaces and are mated to grip tissue as the rollers rotate. Alternatively, protrusions may be teeth or any surface roughness capable of gripping tissue in order to gather it. Rollers 36 and 38 can have bipolar flat-plate electrodes 42 and 44 attached to the flat sides of the rollers with adhesive such as Loctite 3051. Electrodes 42 and 44 can each be about 1 mm thick, about 20 mm long, and about 5 mm wide. Electrodes 42 and 44 may be formed of gold plated brass and soldered to copper wires. Rollers 36 and 38 can be rotated to a position such that electrodes 42 and 44 are substantially parallel to each other, and substantially parallel with respect to a tangent to the outer surface of housing 24 (See for example FIG. 2). In such a position, the adjacent edges of electrodes 42 and 44 can be about 3 mm apart.

Electrodes 42 and 44 can be connected at their narrow ends near conical portion 28 to wires 46 and 48. Wires 46 and 48 can be disposed to pass through the center of rollers 36 and 38 to the proximal end of housing 24. From there, wires 46 and 48 pass along side the endoscope within overtube 26 to the exterior of the patient, where they are connected to a switch and timer control at an external RF generator (not shown). In one embodiment, an RF generator can be provided to deliver a wattage range of from about 15 to about 40 watts at a frequency of between about 300 kiloHertz to 1.5 megaHertz for about 1.3 to 2.0 seconds to ablate a typical varix.

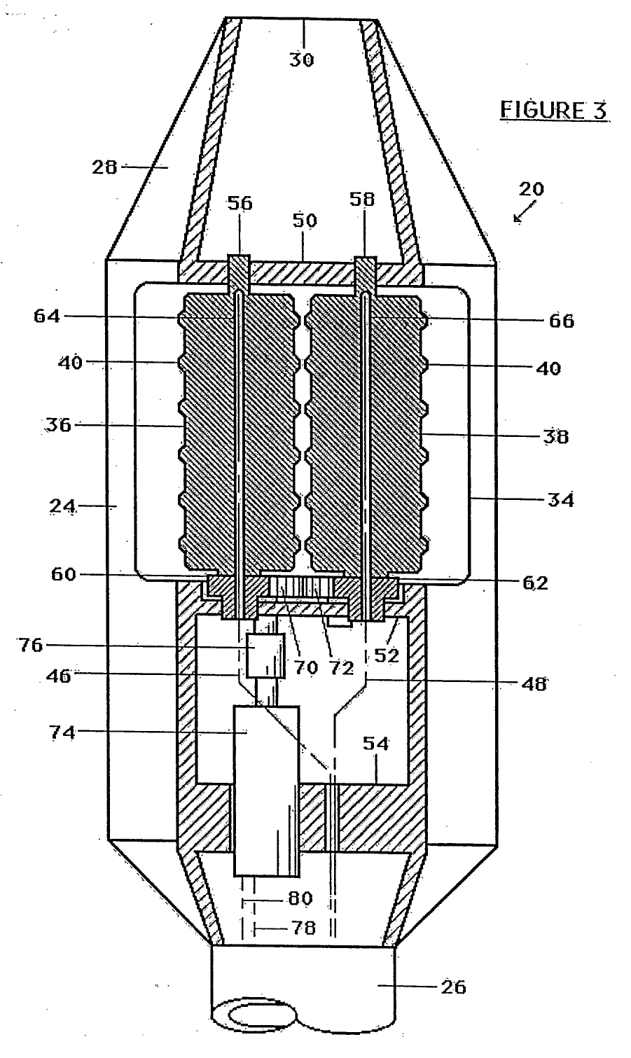

FIGS. 3-6 illustrate how rollers 36 and 38 may be driven with a suitable drive mechanism according to one embodiment. Housing 24 can include wall portions 50, 52, and 54 therein perpendicular to a longitudinal axis of the housing, but which do not interfere with the passage of an endoscope longitudinally through the center of the housing. Rollers 36 and 38 can include axles 56 and 58, which rotate in bushings or recessed holes or slots within wall portion 50. At the opposite ends of rollers 36 and 38 are spur gears 60 and 62 fixedly attached to the rollers. Extending through the centers of rollers 36 and 38 and gears 60 and 62 are wire conduits 64 and 66. The conduits extend from the faces of rollers 36 and 38 perpendicular through axles 56 and 58. By having wires 46 and 48 extend through the centers of the rollers, sufficient wire length is provided to enable the rollers to oscillate 180 degrees without fatiguing the wires as they twist during oscillation of rollers 36 and 38.

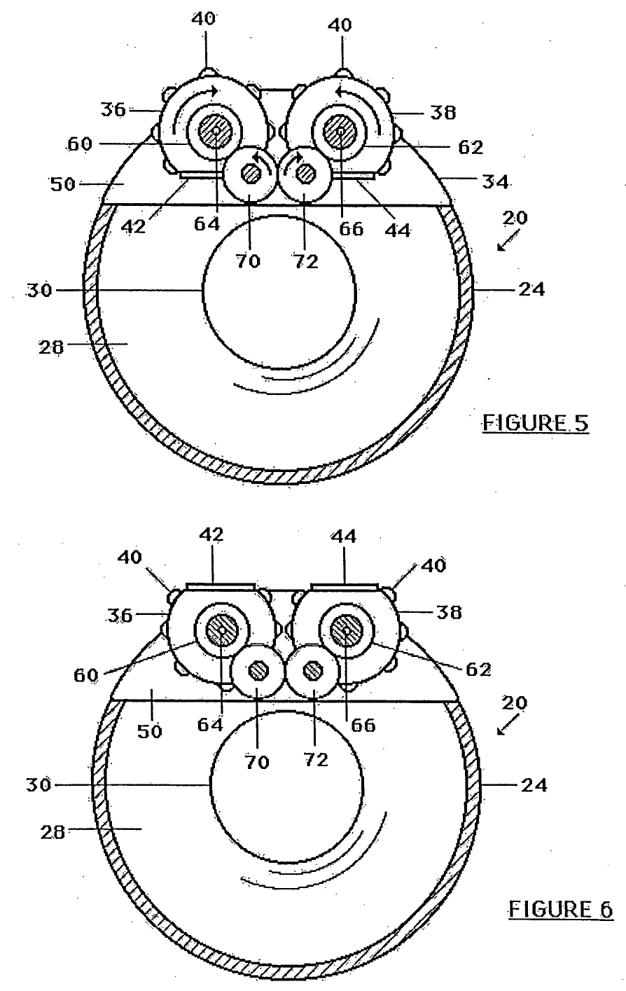

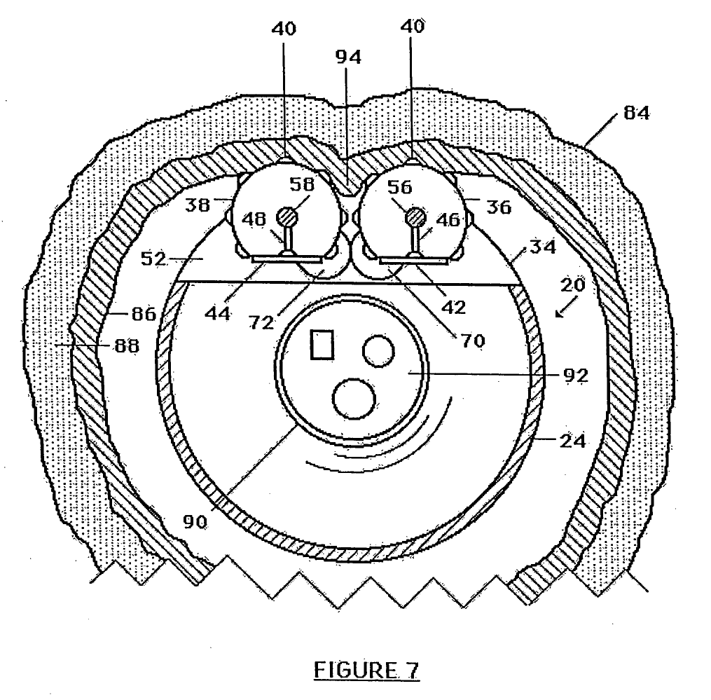

Spur gears 60 and 62 have hubs or shafts which can be supported to rotate in bushings or recess holes or slots within wall portion 52. Gears 60 and 62 can be 32 pitch, 20 tooth gears having a pitchline diameter of about 4.5 mm. Two similarly sized spur gears 70 and 72 can be supported to mesh with gears 60 and 62 to provide the desired rotation of each roller. Gear 70 can be driven through an arc of 90 to 180 degrees between stops, so that rollers 36 and 38 are also rotated 90 to 180 degrees in opposite directions. The two extreme positions of the gears and rollers are shown in FIGS. 5 and 6. In FIG. 3 a drive motor 74 is shown housed in wall portion 54 and its shaft is coupled to the shaft of gear 70 by a coupling 76. Motor wires 78 and 80 (represented by centerlines in FIG. 3) can be routed to pass through the proximal end of housing 24 along side the endoscope to an external power and control source, not shown. Motor 74 can be a 3 mm diameter by 8.1 mm long Faulhaber brushless DC servomotor, part no. BL2S3-025-R-0, available from MicroMo Electronics of Clearwater, Fla. The motor can include a planetary gearhead with a 25:1 gear reduction.

FIG. 4 shows an alternative means for driving spur gear 70. Instead of motor 74, a flexible cable 82 is connected via coupling 76 to spur gear 70. Flexible cable 82 can be about 5 mm in diameter, made of copper with PVC coating such as model MedFlex available from Northwire, Inc. of Osceola, Wis.

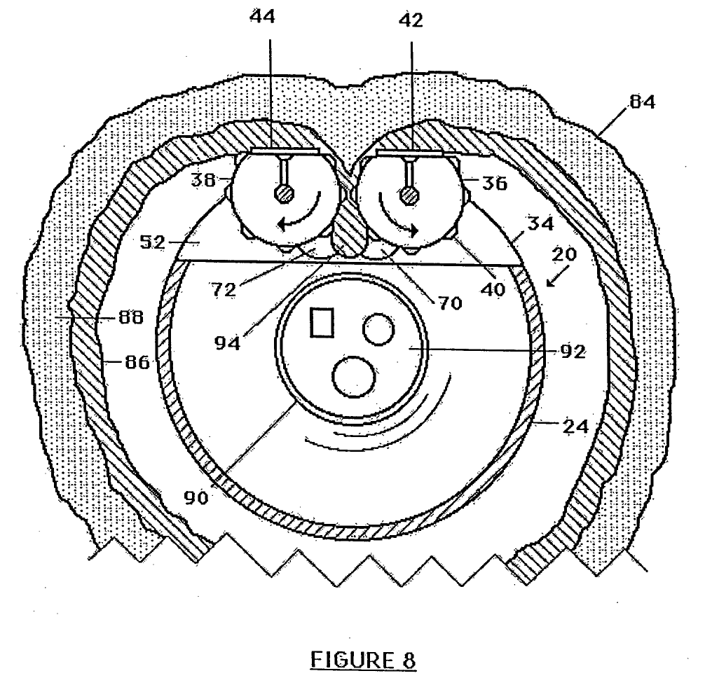

FIGS. 7-10 show a method of operating the present invention to ablate a varix. FIG. 7 shows the inner wall of an esophagus 84, having surface layer 86 and submucosal layer 88. Inserted into esophagus 84 is varix ablation device 20 having an opening 90 with an endoscope 92 centered therein. The sectioned view looking upstream from the distal end of device 20 shows cylindrical housing 24, aperture 34, and rollers 38 and 36. Rollers 36 and 38 have electrodes 42 and 44 connected thereto and wires 46 and 48 soldered to the electrodes, running therefrom into axles 56 and 58. Also seen in housing 24 are spur gears 70 and 72 located at wall 52 of the housing. Rollers 36 and 38 have protrusions 40 which engage surface layer 86 of esophagus 84 around a protruding varix 94 when pressed against layer 86, as shown in FIG. 7.

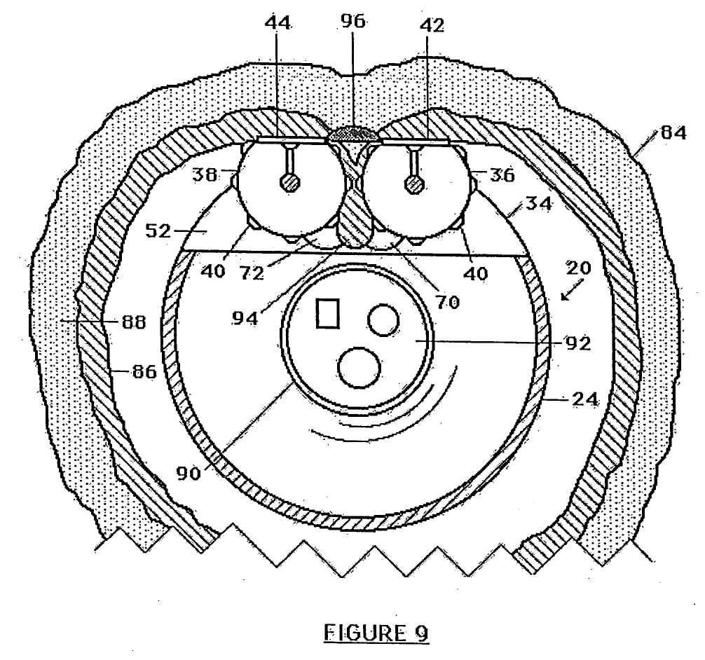

FIG. 8 shows a step of rotating rollers 36 and 38 such that varix 94 and some of layer 86 and submucosal layer 88 are drawn by the grip of protrusions 40 into aperture 34 between rollers 36 and 38. In this step, electrodes 42 and 44 are positioned parallel to each other and tangent to housing 24 with varix 94 held in place between rollers 36 and 38.

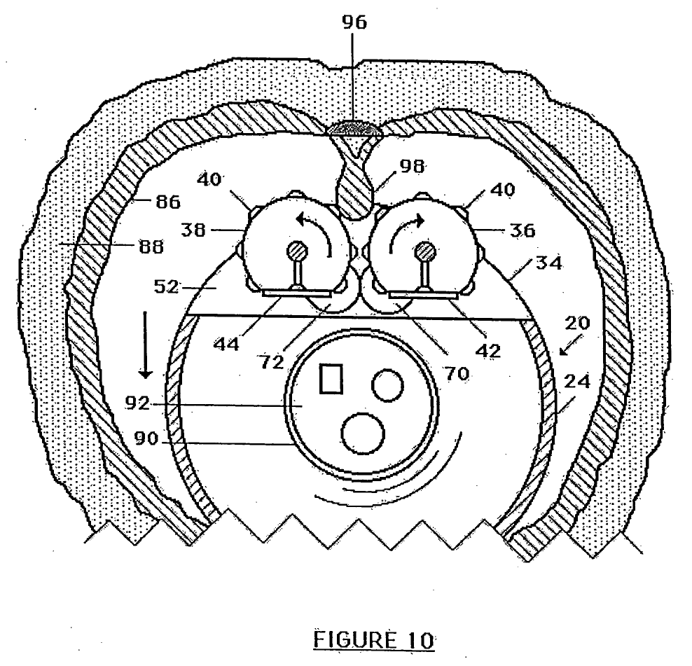

FIG. 9 shows the result of providing RF energy between bipolar electrodes 42 and 44 into tissue at the base of varix 94, thereby creating a cauterized zone 96 primarily within submucosal layer 88, which results in ablated varix 98.

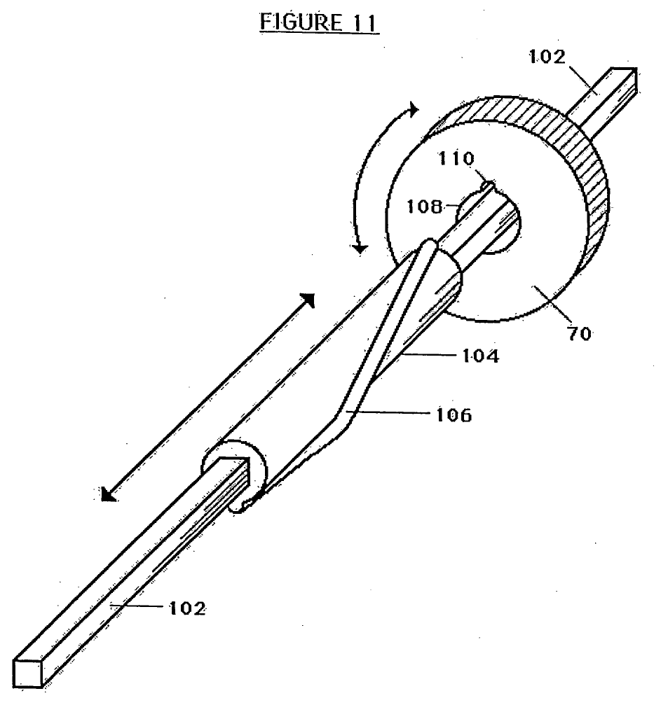

FIG. 10 shows the final step wherein ablation device 20 is retracted from varix 94 while rollers 42 and 44 are reversed 180 degrees to release varix 94 from their grip. Device 20 may now be retracted from esophagus 84 or repositioned to ablate another varix. Ablated varix 98 remains connected to cauterized zone 96 until it either sloughs off or is removed by a snare, not shown, operated within endoscope 92. Alternatively, the device 20 can be provided with a blade or other cutting feature to sever the varix 94, and the severed tissue can be removed (such as by vacuum) through a channel provided in the endoscope.

FIG. 11 shows an alternative means for driving spur gear 70 to rotate rollers 36 and 38 through 180 degrees. An alternative mechanism 100 can includes a slidable spline 102 which can be moved forward and backward (proximally and distally) by any suitable actuator mechanism (e.g. cable/spring) to slide the spline 102 through walls 52 and 54 of housing 24. Fixed to spline 102 is a cylinder 104 having a single worm tooth 106 disposed in helical fashion about the outer surface of cylinder 104. Spur gear 70 can be provided with a hole 108 centered therethrough and an angled key slot 110 extending radially from hole 108.

Key slot 110 is at an angle to the axis of hole 108 to match the angle at which worm tooth 106 wraps around cylinder 104. Thus, when cylinder 104 directed through hole 108, with worm tooth 106 engaged with key slot 110, and with spur gear 70 restrained axially, spur gear 70 will be rotated 180 degrees by worm tooth 106 as the cylinder 104 passes through hole 108.

FIG. 12 illustrates how a varix ablation device may be used with endoscope 92 while positioned within a body cavity. Endoscope 92 fits within a central lumen of an elongated flexible overtube 26 having ablation device 20 at its distal end, and may be held by the hands of a user outside the body. The distal portion of varix ablation device 20 resides within a body cavity, such as esophagus 84 as illustrated in FIG. 12. A body wall 120 is illustrated as an example of a barrier that defines the inside of the body from the outside. The distal portion of varix ablation device 20 resides within the body cavity to treat tissue, and the proximal end remains outside the body to be manipulated by a user. In treatment of the esophagus, the overtube 26, device 20, and endoscope 92 can be introduced through a naturally occurring body orifice, such as by transoral insertion.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

1. A medical device comprising:
a first electrode; and
a second electrode;
wherein the first electrode is movable relative to the second electrode to provide a predetermined desired spacing between an edge of the first electrode and an edge of the second electrode.

2. The medical device of claim 1 wherein the first electrode is rotatable with respect to the second electrode to provide a predetermined desired orientation of the first electrode with respect to the second electrode.

3. The medical device of claim 1 wherein each of the first and second electrodes is rotatable.

4. The medical device of claim 1 wherein the first and second electrodes are positionable in a first position wherein the first and second electrodes are not parallel and positionable in a second position wherein the first and second electrodes are substantially parallel.

5. The medical device of claim 1 wherein the first electrode is disposed on a first rotating member, and wherein the second electrode is disposed on a second rotating member.

6. The medical device of claim 5 wherein at least one of the first and second rotating members comprise a feature for engaging tissue.

7. The medical device of claim 6 wherein at least one of the first and second rotating members comprises a plurality of protrusions which extend from a surface of the rotating member.

8. A medical device comprising:
an elongate, flexible overtube, the elongate flexible overtube having a proximal end and a distal end;
at least two electrodes disposed in association with the distal end of the overtube, wherein at least one of the electrodes is movable with respect to one of the other electrodes.

9. The medical device of claim 8 wherein at least one of the electrodes is movable with respect to another electrode to provide a desired spacing between the electrodes.

10. The medical device of claim 8 wherein each of at least two electrodes are supported to rotate about an axis.

11. The medical device of claim 8 wherein a first electrode and a second electrode are supported for rotation about spaced apart, generally parallel axes, and wherein the first and second electrodes are rotatable to a position wherein the first and second electrodes are substantially parallel.

12. A medical device comprising:
a housing having an opening for receiving tissue therein;
a first tissue engaging member disposed for rotation within the housing;
a second tissue engaging member disposed for rotation within the housing;
a first electrode disposed on the first tissue engaging member; and
a second electrode disposed on the second tissue engaging member.

13. The medical device of claim 1 wherein the first and second tissue engaging members counter rotate with respect to each other.

14. The medical device of claim 1 wherein at least one of the first and second tissue engaging members is rotatable through at least about 90 degrees.

15. The medical device of claim 1 wherein at least one of the first and second tissue engaging members rotates through less than 360 degrees.

16. A method of treating tissue comprising the steps of:
providing a first tissue engaging member having a first electrode;
providing a second tissue engaging member having a second electrode;
moving at least one of the tissue engaging members to provide a predetermined desired spacing between the first and second electrodes; and
energizing the electrodes to treat the tissue.

17. The method of claim 16 comprising the step of rotating at least one of the tissue engaging members to engage tissue.

18. The method of claim 16 comprising counter rotating the tissue engaging members.

19. The method of claim 16 comprising rotating the tissue engaging members through an angle of at least about 90 degrees but less than 360 degrees.

20. The method of claim 16 comprising counter rotating the first and second tissue engaging members to engage tissue prior to energizing the electrodes, and reversing the directions of rotation of the first and second members to disengage tissue after energizing the electrodes.

21. The method of claim 16 comprising rotating the first and second tissue engaging members to position the first and second electrodes in a substantially parallel orientation.
